(19) United States
(12) Patent Application Publication
(10) Pub. No.: US 2016/0095740 A1
(43) Pub. Date: Apr. 7, 2016

(54) METHODS AND DEVICES FOR MONITORING BRUXISM AND/OR SLEEP APNEA AND ALLEVIATING ASSOCIATED CONDITIONS

(71) Applicants: Aris MARDIROSSIAN, Potomac, MD (US); Robert J. SANKER, Rockville, MD (US)

(72) Inventors: Aris MARDIROSSIAN, Potomac, MD (US); Robert J. SANKER, Rockville, MD (US)

(21) Appl. No.: 14/503,733

(22) Filed: Oct. 1, 2014

(51) Int. Cl. A61F 5/56; A61M 16/06; A61M 16/00

(52) U.S. Cl. CPC A61F 5/566; A61M 16/0003; A61M 16/0069; A61M 16/0683; A61F 2005/563; A61M 2016/0027; A61M 2016/003; A61M 2205/3334; A61M 2205/52; A61M 2205/3375

(57) ABSTRACT

Techniques and wearable appliances for monitoring bruxism, and/or for alleviating bruxism or sleep apnea related to bruxism are provided. In certain example embodiments, there is provided a custom teeth guard configured to be attached to at least some teeth in one of the upper or lower jaws, having at least two different non-zero amounts of padding arranged at positions located between teeth in the upper jaw and teeth in the lower jaw in accordance with predetermined strength of grinding of teeth at the positions. The custom teeth guard may be useful in helping to preempt apneic events and/or the like, e.g., by reducing the incidence and/or severity of teeth grinding.

### TECHNICAL FIELD

The disclosure relates generally to bruxism. More particularly, this disclosure relates to wearable devices for monitoring bruxism and/or alleviating bruxism and conditions that occur in relation to bruxism.

### BACKGROUND AND SUMMARY

A large number of patients suffer from bruxism, which is more commonly referred to as teeth grinding. Teeth grinding typically occurs when the patient is asleep, and can cause significant negative effects on the overall health of the patient because, in addition to the excessive wear and tear of the teeth and damage to teeth enamel, it can create headaches and persistent, excessive pain in the jaws.

A variety of “teeth guards” are freely available as purported solutions to teeth grinding. These teeth guards may be known by other names such as “night guard,” “splint,” etc., and they generally protect the teeth by providing a separation between the teeth of the upper jaw and the teeth of the lower jaw. Many of the teeth guards available for purchase at pharmacies, etc., may be custom-fitted to the patient's teeth, for example, by heating (e.g., inserting in boiling water) and wearing so that the guard sets in accordance with the patient's teeth. Yet other teeth guards may be custom fitted based upon molds of patients' teeth acquired by a dental professional.

In certain example embodiments, there is provided a custom teeth guard configured to be attached to at least some teeth in one of the upper or lower jaws, having at least two different non-zero amounts of padding arranged at positions located between teeth in the upper jaw and teeth in the lower jaw in accordance with predetermined strength of grinding of teeth at the positions. The custom teeth guard may be useful in helping to preempt apneic events and/or the like, e.g., by reducing the incidence and/or severity of teeth grinding.

In certain example embodiments, a custom teeth guard is provided. A first part is configured to be attached to at least some teeth in the upper jaw. A second part is configured to be attached to at least some teeth in the lower jaw, with at least one of the first or the second part having at least two different non-zero amounts of padding arranged at positions located between teeth in the upper jaw and teeth in the lower jaw in accordance with predetermined strength of grinding of teeth at the positions. A connector part couples the first part and the second part at the back of the mouth when the first part is attached to the teeth in the upper jaw and the second part is attached to the teeth in the lower jaw. A through hole is provided for breathing.

In certain example embodiments, there is provided a system, comprising: an apparatus with a wearable part configured to be positioned in the mouth of a patient between the upper jaw and the lower jaw and comprising a plurality of pressure sensors to detect grinding pressure of corresponding teeth when teeth in the upper jaw and teeth in the lower jaw contact each other; and circuitry to report, for respective teeth, the detected grinding pressure and an identification of the tooth for which the detected grinding pressure is being reported.

In certain example embodiments, there is provided a system including an apparatus wearable by a patient, comprising: a first part comprising at least one pressure sensor and configured to be positioned in the mouth of the patient between the upper jaw and the lower jaw to detect a pressure event when teeth in the upper jaw and teeth in the lower jaw contact each other; a second part comprising at least one of an airflow sensor configured to detect air flow associated with the throat of the patient, or a vibration sensor configured to detect a vibration event in the throat of the patient; and a processor configured to receive signals from the at least one pressure sensor and at least one of the air flow sensor or the vibration sensor. The processor is further configured to: based upon the detected pressure event and at least one of the detected air flow or the detected vibration event, determine an occurrence of an apnea event; and in response to the determined apnea event, generate a signal to cause at least one of opening the jaw or providing pressurized air to induce breathing.

### BRIEF DESCRIPTION OF THE DRAWINGS

These and other features and advantages may be better and more completely understood by reference to the following detailed description of exemplary illustrative embodiments in conjunction with the drawings, of which:

FIG. 1 is a schematic illustration of an example custom teeth guard in accordance with some embodiments.

FIGS. 2A and 2B are schematic illustrations of an example custom teeth guard with a through hole for breathing, in accordance with some embodiments.

FIG. 3 is a schematic illustration of an example diagnostic device including a part to be worn inside the mouth and a computer, in accordance with some embodiments.

FIG. 4 is a schematic illustration of an example medical device including a part worn inside the mouth, a mask with an attached air injector, and a computer, in accordance with some embodiments.

FIG. 5 is a flowchart of a process for creating a custom teeth guard based upon measured pressure associated with teeth grinding, in accordance with some embodiments.

FIG. 6 is a flowchart of a process for monitoring teeth grinding and apnea, and for causing the alleviation of teeth grinding and/or apnea in real-time, in accordance with some embodiments.

### DETAILED DESCRIPTION OF CERTAIN EXAMPLE EMBODIMENTS

Embodiments of the invention provide custom teeth guards, devices including wearable portions, and methods for monitoring teeth grinding and/or alleviating conditions associated with teeth grinding.

Conventional teeth guards are often inadequate for protecting the teeth of patients who may grind teeth in a manner that exerts varying amounts of pressure in different parts of the mouth, and/or results in a high level of discomfort. Such patients would find that even custom-fitted teeth guards do not provide adequate protection for all teeth. Often such teeth guards are too thin, and unable to protect all teeth, or too thick and cause substantial discomfort when worn.

Some embodiments disclosed herein provide custom teeth guards with varying amounts of padding or thickness arranged in accordance with specific measurements of the pressure levels exerted by respective teeth of the patient. Such teeth guards provide for better protection of the patient's teeth, because extra padding is provided for teeth that exert and/or is subject to higher amounts of pressure and less or no padding is provided for teeth that exert and/or is subject to little or no pressure.

Some embodiments provide a diagnostic device that can be worn by the patient inside the mouth, that include pressure sensors for respective teeth in order to measure the amount of pressure each tooth exerts and/or is subjected to. The pressure measurements can be used for forming a custom teeth guard with amounts of padding for respective teeth varied in accordance with the predetermined pattern of pressure. In some embodiments, the diagnostic device may include a vibration sensor and/or air flow sensor that can, simultaneously with the pressure sensing, detect whether an apnea event is occurring.

Sleep apnea may be related to bruxism, and evidence suggests that treating sleep apnea can help alleviate bruxism. One potential cause of bruxism is the instability of airways during sleep. Upper Airway Resistance Syndrome and Obstructive Sleep Apnea are conditions related to blocked airways. As a patient falls asleep, his/her muscles about the upper airway, which is made up of muscles and soft tissue, relax and the tongue falls back in the direction of the back of the airway or throat. For some patients, this can lead to significant upper airway obstruction during sleep. Clenching teeth appears to be a natural reaction to this obstruction. When the patient clenches or grinds teeth, the tongue may be moved away from the back of the upper airway or throat, thereby releasing the obstruction at least temporarily. Evidence also suggests that bruxism might cause apnea. For instance, if a person's nasal cavities are blocked and the person is used to mouth breathing, bruxism could cause the temporary or complete cession of breathing.

When an apnea event is detected simultaneously with a teeth grinding event, some embodiments provide for alleviating the apnea condition by a technique such as delivering a vibration to the patients jaw and or causing the delivery of pressurized gas in order to induce breathing. Alternatively, or in addition, the likelihood of apneic event occurring can be reduced and apneic events can be prevented (instead of compensated for) ahead of time. In other words, the inventors have observed that teeth grinding can cause certain apneic events. Thus, by reducing the impact of teeth grinding, it is in essence possible to sometimes preempt apneic events from occurring in the first place.

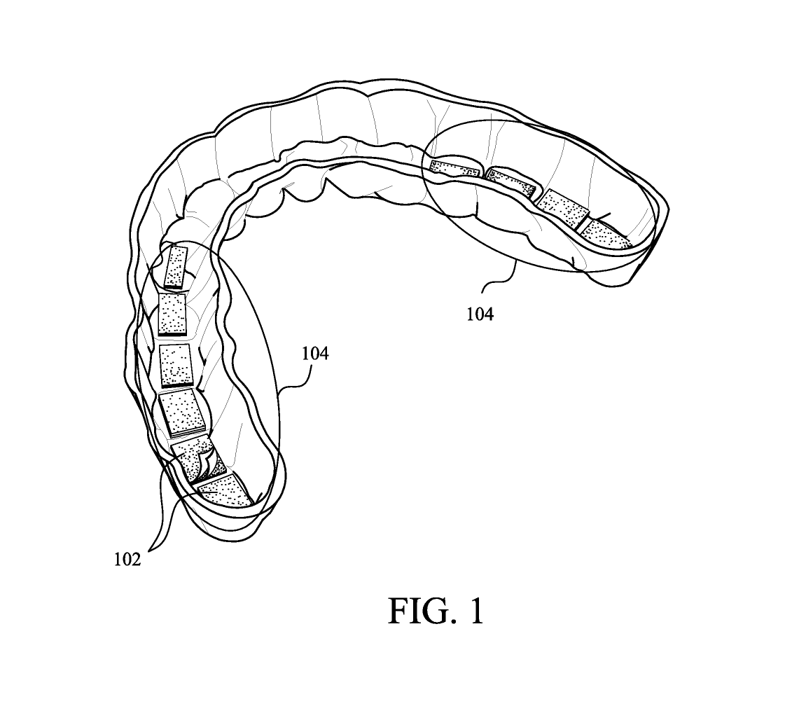

FIG. 1 is a schematic illustration of an example custom teeth guard 100 in accordance with some embodiments. Teeth guard 100 is shaped and sized to fit a portion of the upper jaw or lower jaw. In some embodiments, teeth guard 100 is custom fitted to the patient either using a mold to create the teeth guard, or by using a technique such as “boil and mold” to better fit the patient. Teeth guard 100 may be configured to cover all of the teeth of one of the upper or lower jaw, or only some teeth of that jaw. The illustrated example teeth guard 100 is configured to cover 14 teeth distributed generally symmetrically from the front (e.g. in effect, the illustrated teeth guard 100 is not specifically configured to attach to the rear-most tooth on each side, when the patient has 16 teeth in each jaw). The teeth guard 100 may be made using a material such as, but not limited to, rubber, silicone, plastic, and/or any combination of thereof

Teeth guard 100 includes padding 102 in areas 104 that are located between the teeth of the upper and lower jaws. The amount of padding for each tooth may be determined separately. For example, in embodiments, the amount of padding may be custom determined for the patient based upon measurements of the teeth grinding pressure. In some embodiments, teeth guard has two or more non-zero levels of padding. For typical patients, the number of unique padding levels may extend from 1 where all teeth have the same amount of padding to 16 where each tooth exerts a unique grinding pressure amount and is therefore provided with a unique padding amount. In some embodiments, a range of measured pressure values may be assigned to a particular padding amount. The padding may include sealed liquid or air filled layers. In some embodiments, the padding may include a cushion of a material such as silicon, rubber, nylon or other material suitable for withstanding a desired amount of bite pressure. The padding for a tooth may cover the biting surface of the tooth including the edges. According to some embodiments, the different amounts of padding is provided for individual teeth by inserting different numbers of padding layers, where each layer provides a uniform amount of padding, for individual teeth in accordance with the determined pressure pattern.

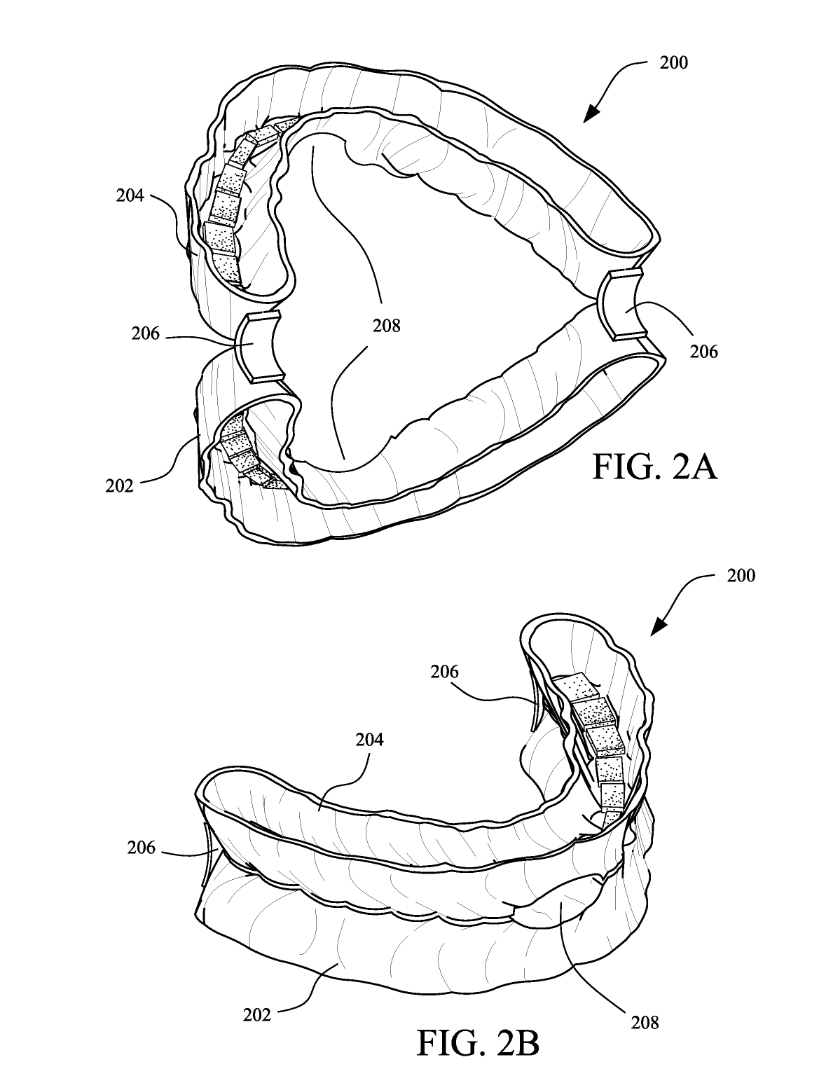

FIGS. 2A and 2B are schematic illustrations of an example custom teeth guard 200 with a through hole for breathing, in accordance with some embodiments. Teeth guard 200 includes a first part 202 attachable to the teeth of the lower jaw, and a second part 204 attachable to the teeth of the upper jaw.

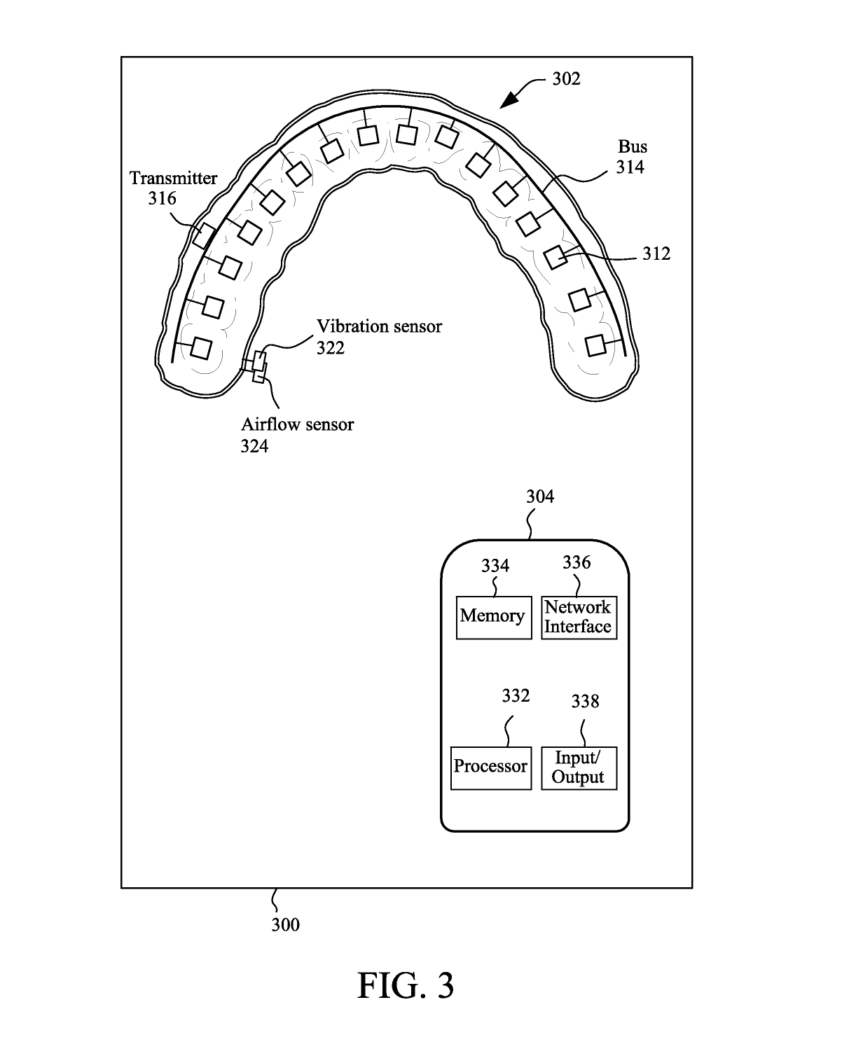

Each of the first and second parts may include padding arranged in accordance with customized pressure profile for the wearer. The customized pressure profile may have been predetermined based upon actual teeth grinding pressure measurements. Teeth grinding pressure measurements may be obtained, for example, by using a sensor-equipped teeth guard as described, for example, in relation to FIG. 3.

The first part and the second part of teeth guard 200 may be connected by a connecting piece 206 located at the back of the mouth. The connecting piece 206 may be constructed from rubber, silicon, or similar material.

One or both of the first part and the second part of the teeth guard may include at least one through-hole 208 to facilitate breathing. In the illustrated embodiment, through-hole 208 is formed by forming a portion of the opening in the first part and another part of the opening in the second part, such that when the teeth guard is being worn by a patient and the jaws are closed, a through-hole having a substantially circular opening formed in the front so that breathing through the mouth is facilitated. Embodiments may include through-holes in various numbers, locations, sizes and/or shapes, in the teeth guard.

FIG. 3 is a schematic illustration of an example diagnostic device 300 including a part worn inside the mouth, and a computer, in accordance with some embodiments. Diagnostic device 300 may be worn by a patient in order to measure his or her specific teeth grinding patterns. Diagnostic device 300 may also be used for detecting when an apnea event occurs simultaneously with a teeth grinding event. Diagnostic device 300 may further be used in alleviating teeth grinding and/or apnea and other conditions that occur simultaneously with teeth grinding.

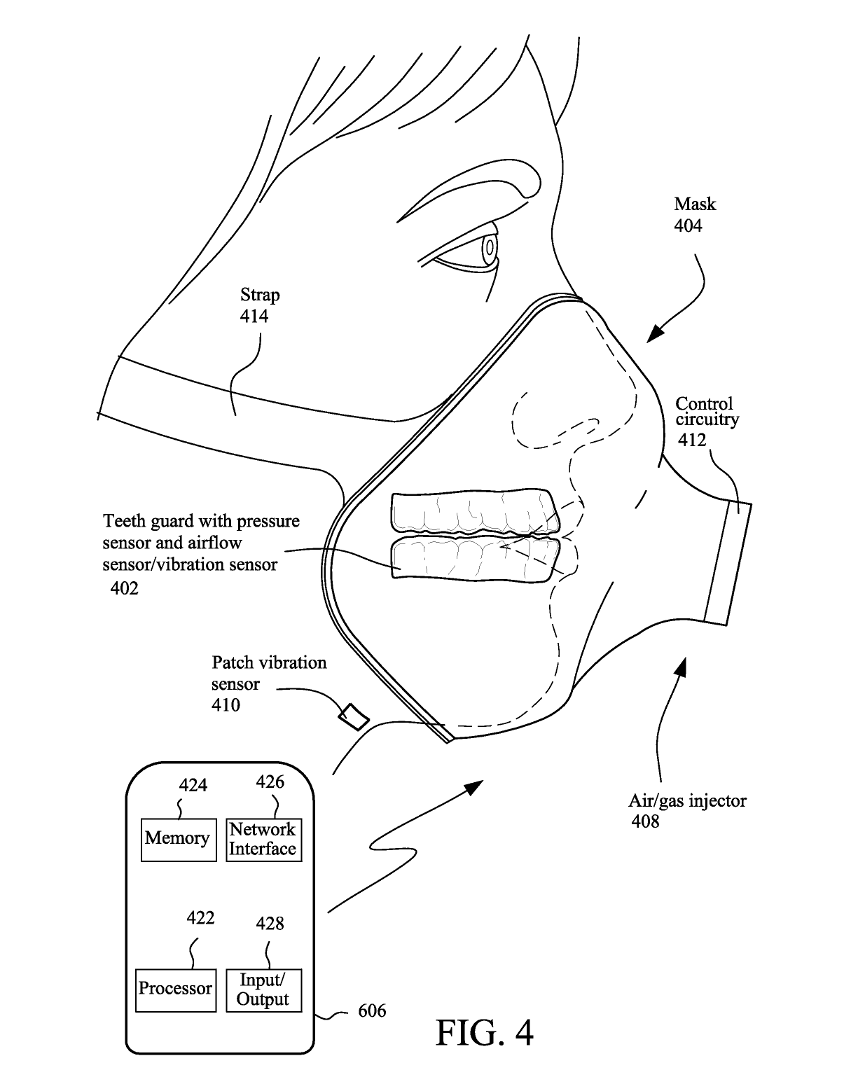

Diagnostic device 300 includes a wearable part 302 and a computer 304. The wearable part 302 is sized and shaped to fit at least some teeth of either the upper jaw or the lower jaw. Wearable part 302 may include separate pressure sensors 312 for each of a plurality of teeth. Each of any number of the teeth may be provided with its own pressure sensor capable of measuring the pressure associated with that individual tooth. Suitable pressure sensors are further described in relation to FIG. 4 below.

The measurements of the pressure sensors 312 are transferred over a bus 314 to a transmitter 316. Each pressure sensor may transmit information including a measured pressure, a time associated with the measurement, and an identification of the tooth.

In some embodiments, wearable part 302 may also have attached an air flow sensor 322 and/or a vibration sensor 324. Suitable air flow sensors and vibration sensors are further described in relation to FIG. 4 below. The measurements obtained by the air flow sensor 322 and/or vibration sensor 324 may also be transferred over bus 314 to transmitter 316.

Transmitter 316 operates to receive the readings of the pressure sensors 312, air flow sensor 322 and/or vibration sensor 324, and to transfer the received readings to a computer 304. Transmitter 316 may include Bluetooth, Wi-Fi, or like wireless data transfer technology. In some embodiments, transmitter 316 may include transfer of data over wired connections. The sensors may be powered by individual batteries, or one or more batteries may provide power to all devices incorporated with the teeth guard. The batteries are of sizes suitable for including in the small confines of the teeth guard and/or the sensors. In some embodiments, other sources of power, for example, using saliva and/or teeth movement/grinding may be used to supplement or replace battery power.

Computer 304 may include a processor 332, a memory 334, a network interface 336, and input/output 338. In some embodiments, computer 304 is remotely located from the wearable part 302. In some other embodiments, computer 304 is integrated in the wearable part 302. Computer 304 operates to receive the measurements from various sensors, and to develop a teeth grinding pressure profile for a patient. A pressure profile may include a frequency chart indicating the amount of pressure exerted by or exerted upon each of a plurality of teeth. The profile may be constructed in a manner that enables identifying a plurality, or more than two, levels of pressure measurements. The profile may subsequently be used to determine a level (or amount) of padding to be provided for each individual tooth. According to an example, for each level of pressure measurement identified in the pressure profile, a corresponding amount of padding may be determined.

The profile also may be cross-referenced with detected apneic events, e.g., to determine whether the incidence of teeth grinding is associated with (e.g., likely a contributing factor to) the cessation of breathing associated with apneic events. For instance, the airflow sensor data may be used to 324 may provide input in this regard. Vibration sensor 322 also may be used to provide useful information, as snoring is sometimes associated with sleep apnea or the like. The profile thus may be a useful tool in assessing possible root causes for apneic events and the like.

Some embodiments may include a software app, e.g., executing on a portable device such as a smartphone, smartwatch, tablet, or other handheld electronic device, which communicates with a teeth guard being worn by a patient. The app may, assuming that it is executing in a smartphone, use a Bluetooth, WiFi, 4G or other communication connection of the electronic device to receive signals from the sensors in the teeth guard. As noted above the teeth guard according to embodiments, can monitor and transmit pressure information for individual teeth. Upon determining that a certain pattern and/or certain level of bruxism is occurring, the app may use the smartphone's audio recording features and microphone to turn on audio recording so that the patient's snoring may be captured. The app may be configured such that recording is turned off after a predetermined interval, or when either the snoring drops below a predetermined threshold or the signals from the teeth guard sensors indicate that teeth grinding has reduced to a level below a predetermined threshold. The bruxism information received from the teeth guard sensors and snoring information received from the recorded audio can be stored on the smartphone or external memory, used in further analysis or processing, or transmitted to an external location for further processing and/or monitoring. For example, such information may be logged over intervals for diagnostic purposes, and according to another example, such information can be used to trigger other types of monitoring such as an electro cardiogram (ECG) etc.

FIG. 4 illustrates an example medical device 400 including a part 402 worn by a patient inside the mouth, a mask 404 with an attached air injector 408 and a computer 406, in accordance with some embodiments. Medical device 400 may be used, for example, for detecting and relieving teeth grinding and/or teeth grinding related apnea.

Medical device 400 may be worn by the patient by inserting the teeth guard 402 into the mouth, and then fastening the mask 404 by a strap 414 to the face. The mask 404 and the strap 414 are shaped and configured such that the device 400 can be comfortably worn by the patient to sleep.

According to some embodiments, teeth guard 402 is sized and configured to be attached to the upper jaw, and provides a barrier to separate a plurality of teeth of the upper jaw from corresponding teeth of the lower jaw. In some other embodiments, teeth guard 402 is sized and configured to be attached to the lower jaw. The teeth guard 402 may encompass each tooth in two directions and on the top, and remains open in the direction of the tooth enamel. The teeth guard may be constructed from materials including, for example, and without limitation, plastic, rubber, and/or silicone.

Teeth guard 402 includes a plurality of pressure sensors, embedded in the barrier between teeth of the upper and lower jaws, which operate to sense the amount of pressure exerted by each tooth. The pressure sensors may be of the same type or of more than one type. Many types of pressure sensors are suitable for use in the teeth guard. For example, piezoresistive, capacitive, electromagnetic, or piezoelectric types of pressure sensors may be used.

Teeth guard 402 may include a transmitter (not shown in FIG. 4) that transmits the pressure measurements obtained by pressure sensors to computer 406. The pressure measurements may be provided to the transmitter via a wired channel. In some embodiments, at least some of the pressure sensors may transmit pressure readings wirelessly.

In some embodiments, teeth guard 402 may include an airflow sensor to sense the airflow to/from the patient's mouth and/or a vibration sensor to sense the vibrations of the patient's throat. The airflow sensor and/or vibration sensor may be attached to the teeth guard 402 in proximity to the throat opening, such that, accuracy of the airflow measurements and/or the throat-originated vibrations are improved. Any type of air flow sensor that is of a sufficiently small size to fit in or to the teeth guard 402, and is capable of measuring a breath level in the mouth may be used. Any type of vibration sensor that is of a sufficiently small size to fit in or to the teeth guard 402, and is capable of measuring a vibration generated in the throat during apnea may be used. Microphones (e.g., with good low frequency sensitivity and associated optional low frequency band pass filters) may be used in this regard. The air flow sensor measurements and/or the vibration sensor measurements may be transmitted to computer 406 by a transmitter included in the teeth guard 402. Pressure measurements, airflow measurements, and vibration measurements may be transmitted to computer 406 by the same or different transmitters in teeth guard 402.

Mask 404 may be made of rubber, plastic, silicone, any other suitable material, or any combination thereof. Mask 404 is sized, shaped, and configured such that it fits comfortably and preferably in an airtight manner, to the patient's face when secured by a strap. The strap 414 can be constructed from elastic or other material that stretches to tightly but comfortably secure the mask to the patient's face.

Mask 404 may have built in, or attached securely thereto in a replaceable manner, a pressurized gas injector 408. Pressurized gas injector 408, for example, may be positioned in the mask such that it may inject air directly into the patient's mouth. In some embodiments, a tube extending from the pressurized gas injector 408 is attached to teeth guard 404. The tube is preferably sized, and is of such construction, such that it does prevent the patient from closing his or her mouth while the tube is inserted in the mouth.

Pressurized gas injector may include control circuitry 412 that operates to control the time when gas injection occurs and also the amount of gas that will be injected. In some embodiments, control circuitry 412 receives instructions from computer 406 and accordingly causes injection of gas from pressurized gas injector 408. The contents of the pressurized gas injector 408 may include oxygen or other mix of air including oxygen. Control circuitry 402 may include further features, such as, for example, tracking and/or indicating the amount of remaining pressurized gas. Suitable pressures may be in the 2-20 cmH₂O range in certain example embodiments. The precise pressure of the breathable gas may vary on inhalation and exhalation, e.g., to reduce patient breathing effort. Feedback also may be provided such that detected teeth grinding and airflow restriction through the mouth triggers a decrease in the flow and/or pressure delivered through the mouth and optionally increases the flow and/or pressure delivered through the nose (e.g., using the same or separate mask appliance(s)).

In some embodiments, a vibration sensor 410 may be positioned remotely from the teeth guard, at a location of the neck of the patient. Vibration sensor 410 is positioned so that throat vibrations can be sensed. The throat vibrations, for example, may be related to (e.g., caused by, indicative of, or otherwise associated with) apnea. Vibration sensor 410 may be worn as a patch (e.g. stick-on patch), or strapped on, at an appropriate location of the neck. Vibration sensor 410 may include a transmitter configured to transmit the sensed vibrations to computer 406. Power to the sensors may be obtained from one or more batteries, and/or other sources, as described in relation to FIG. 3 above. Power to the gas injector may also be provided by batteries. However, in some embodiments, the power to the gas injector and associated circuitry may be obtained from an AC power source. In certain example embodiments, a remote blower may be provided, and the blower may be plugged into a standard wall socket. In certain example embodiments, the remote blower may house control circuitry for receiving, analyzing, and/or optionally relaying to a remote computer system for physician analysis data from the patch vibration sensor 410, pressure sensor 402, etc.

In this regard, in certain example embodiments, computer 406 includes a processor 422, a memory 424, a network interface 426, and an input/output 428. Computer 406 operates to receive, via network interface 426, the pressure measurements from pressure sensors embedded in the teeth guard 402, and measurements from other sensors such as any air flow sensor or vibration sensor that may also be included in the teeth guard 402. In some embodiments, computer 406 is located remotely from teeth guard 402. In some other embodiments, computer 406 may be incorporated in mask 404, control circuitry 412, or teeth guard 402.

Processor 422 may include one or more processors such as, but not limited to, central processing unit (CPU), application specific integrated circuits (ASIC), or other processor capable of executing a sequence of program logic instructions. Memory 424 may include any type of dynamic RAM (DRAM), static RAM (SRAM), flash memory, or a combination thereof. In some embodiments, such as when computer 406 is a standalone computer located remotely from teeth guard 402, memory 424 may include magnetic disk, optical disk, or other types of memories. Network interface 426 operates to receive information including, for example, measurements from the various sensors, and to transmit information including, for example, instructing control circuitry 412 to inject air, instructing a small vibration device in the teeth guard to deliver a vibration, etc.

Computer 402 processes the received measurements from various sensors, and, based upon the received measurements, determines whether a teeth grinding and/or an apnea with teeth grinding is occurring. According to a determination that a teeth grinding and/or an apnea event is occurring, the computer may instruct the delivery of pressurized gas and/or a vibration to the jaw/teeth in order to relieve, at least temporarily, the apnea condition and/or the teeth grinding. During sleep, it is thought that the jaws and throat muscles relax, causing obstructive sleep apnea. It is further thought that a patient may begin to grind teeth in response to the onset of obstructive sleep apnea. Alternatively, or in addition, teeth grinding might sometimes cause a temporary and/or complete cessation of breathing, thereby triggering hypopnea and/or apnea.

Input/output interface 428 provides for configuring medical device 400. Configurations may include, for example: a tooth pressure threshold which, when exceeded, results in a determination that a teeth grinding event has occurred; air inflow and/or air outflow thresholds that, when exceeded, results in a determination that an apnea event has occurred; and a throat vibration threshold that, when exceeded, results in a determination that an apnea event has occurred. In some embodiments, more than one threshold may be set for one or more types of measurements. Configurations may also include an amount of gas to inject each time a gas injection is instructed, an intensity and/or duration of a vibration delivered to the jaw, etc. Other configurations may include, but are not limited to, parameters for frequency information collection (e.g., for generation of frequency graphs, etc.) for pressure measurements, airflow measurements, and/or throat vibration measurements. In some embodiments, computer 406 may output frequency information and/or timing information associated with the received pressure, airflow and/or vibration measurements.

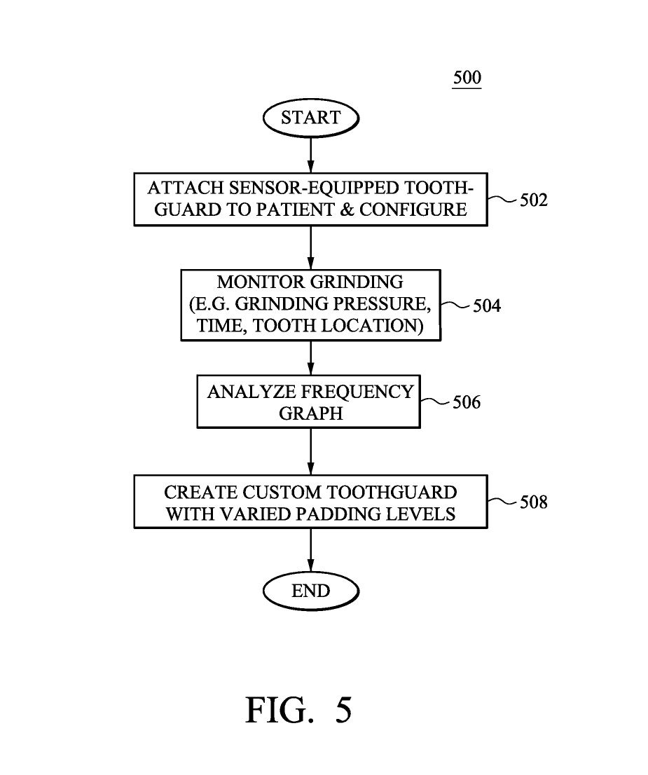

FIG. 5 illustrates a flowchart for a process 500 for creating a custom teeth guard based upon pressure measurements associated with teeth grinding, in accordance with some embodiments. Process 500 may, for example, be performed by a computer such as computer 304 shown in FIG. 3.

After entering process 500, at operation 502, a sensor-equipped teeth guard is worn by a patient. The sensor-equipped teeth guard may, in an embodiment, include teeth guard 300 illustrated in FIG. 3. The teeth guard may be worn by attaching it to the upper jaw or lower jaw teeth. As described in relation to FIGS. 3 and 4, the teeth guard may provide for monitoring the pressure on any plurality of the patient's teeth. The monitoring system may be configured by, for example, providing thresholds detecting grinding pressure on tooth, teeth for which pressure information is to be collected, etc. After the teeth guard is safely worn by the patient and the system is configured, the monitoring may be activated.

After the monitoring is activated, at operation 504, teeth pressure is monitored for one or more teeth in accordance with configurations. The pressure measurements received may include a grinding pressure, time (e.g. time initially detected), and the location of the tooth for which the measurement is taken. The grinding pressure may be indicated using any units. The time may be indicated as the time at which the measurement was taken. The location of the tooth may be indicated by an identifier for the tooth, such as, for example, a unique identifier provided to each pressure sensor included in the teeth. The received measurements may be stored in a memory for the duration of the monitoring process.

At operation 506, which may occurs during or after the monitoring process, a frequency graph of the received pressure measurements is constructed and analyzed. According to an embodiment, the frequency graph indicates how often and how severely pressure has been imposed on the monitored plurality of teeth.

At operation 508, based upon the information obtained from the frequency graph of teeth pressure for the patient, a custom teeth guard is constructed. The padding amount in the teeth guard provided to respective teeth may be changed in accordance with the actual pressure measurements indicated for the respective teeth in the frequency graph. For example, a highest pressure measurement may cause a corresponding tooth to be provided 3 layers of padding, and a medium level pressure measurement may cause the corresponding teeth to be assigned 1 level of padding.

Thus, at operation 508, a custom teeth guard is formed, where the amount of padding for respective teeth has been changed in accordance with actual teeth grinding pressure of the patient.

Process 500 may terminate after operation 508.

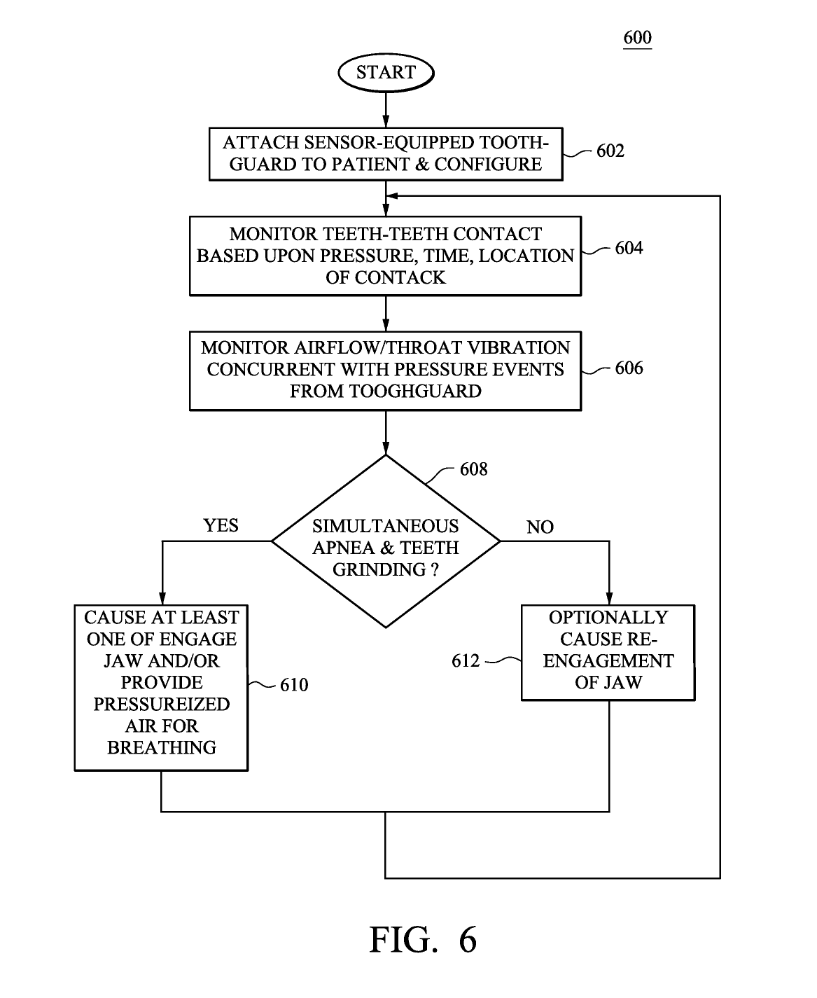

FIG. 6 is a process for monitoring teeth grinding and apnea events, and for causing the alleviation of apnea in real-time, in accordance with some embodiments. Process 600 may, for example, be performed by a computer such as computer 304 shown in FIG. 3 or computer 406 shown in FIG. 4.

After entering process 600, at operation 602, a sensor-equipped teeth guard is worn by a patient. The sensor-equipped teeth guard may, in an embodiment, include teeth guard 300 illustrated in FIG. 3. The teeth guard may be worn by attaching it to the upper jaw or lower jaw teeth. As described in relation to FIGS. 3 and 4, the teeth guard may provide for monitoring the pressure on any plurality of the patient's teeth. The monitoring system may be configured by, for example, providing thresholds detecting grinding pressure on tooth, teeth for which pressure information is to be collected, etc. According to some embodiments, the patient may also be provided with a mask equipped with an air injector, such as that shown in FIG. 4, which the patient wears over the mouth, and/or a vibration sensor, such as vibration sensor 410, located remotely from the teeth guard. After the teeth guard (and in some embodiments, the mask) is safely worn by the patient and the system is configured, the monitoring may be activated.

After the monitoring is activated, at operation 604, teeth pressure is monitored for one or more teeth in accordance with configurations. The pressure measurements received may include a grinding pressure, time (e.g., time initially detected), and the location of the tooth for which the measurement is taken. The grinding pressure may be indicated using any units. The time may be indicated as the time at which the measurement was taken. The location of the tooth may be indicated by an identifier for the tooth, such as, for example, a unique identifier provided to each pressure sensor included in the teeth. The received measurements may be stored in a memory for the duration of the monitoring process.

At operation 606, the received measurements are continuously analyzed in real-time to determine the occurrence of teeth grinding events and apnea events. The computer may be preconfigured to detect a teeth grinding event if a certain number of teeth (and/or particular teeth) indicate pressure levels beyond a configured threshold. The computer may be preconfigured to also detect a potential apnea event based upon a predetermined low level of air flow measured by an air flow sensor, and/or a vibration level that exceeds a predetermined threshold amount.

At operation 608, when a teeth grinding event and an apnea event are detected simultaneously or near simultaneously, the computer determines that action is due for alleviating the apnea condition and/or the teeth grinding. Then, at operation 610, the computer instructs a vibration device to deliver a vibration to at least one of the jaws in order to re-engage the jaw which may have relaxed earlier, thereby alleviating the apnea condition and also the teeth grinding. In the same or other embodiments, operation 610 may also include causing pressurized gas to be injected into the mouth, thus opening the airways and alleviating the apnea condition, and possibly, also the teeth grinding. Whereas some embodiments may include one or the other of either the delivery of the vibration to the jaw, or the delivery of pressurized air to the mouth, some embodiments may include both. In yet other embodiments, the apnea condition and/or the teeth grinding may be alleviated at operation 608 by using a technique other than the delivery of the vibration to the jaw or the delivery of pressurized air to the mouth.

If, at operation 608, if simultaneous or near-simultaneous apnea and teeth grinding are not detected then, optionally at operation 612, for example, if a teeth grinding is detected, the computer may cause the jaw to be re-engaged to alleviate the teeth grinding. For example, the jaw may caused to be re-engaged by providing a vibration signal to the jaw.

Process 600 may continue to be performed until deactivated by the patient. For example, having activated the process prior to sleeping, the patient may deactivate the system upon awakening from the sleep.

It will be appreciated that the example techniques set forth herein may use sensors to detect muscle contractions suggestive of bruxism. This may be used to confirm data gathered from tooth sensors and/or obtain gross measurements that trigger data measurements from the individual tooth sensors. It will be appreciated that the example techniques set forth herein are advantageous in the sense that they are very accurate in determining where and when bruxism is occurring, e.g., on a tooth-by-tooth basis. Such techniques may lead to improved diagnosis because of the higher-quality data captured, improved treatment because of the ability to manufacture better or more customized guards, etc.

Although certain example embodiments have been described in relation to apneas, it will be appreciated that the techniques described herein may be useful in diagnosing, treating, or otherwise handling hypopneas as well.

Although preferred embodiments of the disclosure have been described, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses as well as obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein made should not, in any way, be limited by or to the particular disclosed embodiments described above.

What is claimed is:

1. A custom teeth guard configured to be attached to at least some teeth in one of the upper or lower jaws, having at least two different non-zero amounts of padding arranged at positions located between teeth in the upper jaw and teeth in the lower jaw in accordance with predetermined strength of grinding of teeth at the positions.

2. The custom teeth guard according to claim 1, wherein the strength of grinding of teeth at the positions is predetermined based upon actual measurements of pressure.

3. A custom teeth guard, comprising:
a first part configured to be attached to at least some teeth in the upper jaw;
a second part configured to be attached to at least some teeth in the lower jaw, at least one of the first or the second part having at least two different non-zero amounts of padding arranged at positions located between teeth in the upper jaw and teeth in the lower jaw in accordance with predetermined strength of grinding of teeth at the positions;
a connector part coupling the first part and the second part at the back of the mouth when the first part is attached to the teeth in the upper jaw and the second part is attached to the teeth in the lower jaw; and
a through hole for facilitating breathing.

4. The custom teeth guard according to claim 3, wherein the strength of grinding of teeth at the positions is predetermined based upon actual measurements of pressure.

5. A system, comprising:
an apparatus with a wearable part configured to be positioned in the mouth of a patient between the upper jaw and the lower jaw and comprising:
a plurality of pressure sensors to detect grinding pressure of corresponding teeth when teeth in the upper jaw and teeth in the lower jaw contact each other; and
circuitry to report, for respective teeth, the detected grinding pressure and an identification of the tooth for which the detected grinding pressure is being reported.

6. The system of claim 5, further comprising:
a memory; and
a processor configured to receive the report and to store the detected grinding pressure and the identification of the tooth in the memory.

7. The system of claim 5, wherein the circuitry further reports a time associated with the contact, and wherein the apparatus further comprises:
a memory; and
a processor configured to receive the report and to store the detected grinding pressure, the time associated with the contact and the identification of the tooth in the memory.

8. The system of claim 7, wherein the processor is further configured to, using the grinding information stored in the memory, generate a frequency graph showing the frequency of grinding, the pressure associated with the grinding, and the time associated with the grinding.

9. A system including an apparatus wearable by a patient, comprising:
a first part comprising at least one pressure sensor and configured to be positioned in the mouth of the patient between the upper jaw and the lower jaw to detect a pressure event when teeth in the upper jaw and teeth in the lower jaw contact each other;
a second part comprising at least one of an airflow sensor configured to detect air flow associated with the throat of the patient, or a vibration sensor configured to detect a vibration event in the throat of the patient; and
a processor configured to receive signals from the at least one pressure sensor and at least one of the air flow sensor or the vibration sensor, and further configured to:
based upon the detected pressure event and at least one of the detected air flow or the detected vibration event, determine an occurrence of an apnea event; and
in response to the determined apnea event, generate a signal to cause at least one of opening the jaw or providing pressurized air to induce breathing.

10. The system according to claim 9, wherein the determining an occurrence of an apnea event comprises:
a first determining operation indicating whether the detected pressure event includes a pressure measurement above a pressure threshold;
a second determining operation indicating whether the detected vibration event includes a vibration measurement above a vibration threshold;
a third determining whether times of occurrence for the detected pressure event and the detected vibration event are within a specified time interval; and
based upon the first, second and third determining, performing said determining that the apnea event occurred.

11. The system according to claim 9, wherein the generated signal is a signal for causing the upper and/or lower jaw to open so as to engage muscles at the back of the throat.

12. The system according to claim 11, further comprising a vibration device configured to be positioned in contact with a lower portion of the patient's face, wherein the signal for causing the upper and/or lower jaw to open causes vibration of the vibration device.

13. The system according to claim 11, further comprising a pressurized gas dispenser configured to be positioned in the mouth, wherein the signal for causing the upper and/or lower jaw to open causes pressurized gas dispenser to eject liquid to the mouth.

14. The system according to claim 13, wherein the signal for causing the upper and/or lower jaw to open is received wirelessly from the processor.

15. The system according to claim 13, wherein the pressurized gas dispenser is removably attached to a face mask worn by the patient.

16. The system according to claim 13, wherein the processor is further configured to, in response to the determined apnea event, generate a signal to temporarily cease providing pressurized air via the mouth when teeth grinding is detected.

17. The system according to claim 13, wherein the processor is further configured to, in response to the determined apnea event, generate a signal to temporarily begin providing pressurized air via the nose when teeth grinding is detected.

18. The system according to claim 9, further comprising a microphone, wherein a processor is configured to, based upon at least the detected pressure event, enable recording of a patient's snoring.

19. The system according to claim 18, wherein the recorded snoring is stored in a memory in association with the detected pressure event.
